(19) United States
(12) Patent Application Publication
(10) Pub. No.: US 2012/0214775 A1
(43) Pub. Date: Aug. 23, 2012

(54) Pharmaceutical Salts of 3-O-(3',3'-Dimethylsuccinyl) Betulinic Acid

(75) Inventors: Martin Dale POWER, Overland Park, KS (US); David Eugene MARTIN, Gaithersburg, MD (US)

(73) Assignee: Myrexis, Inc., Salt Lake City, UT (US)

(21) Appl. No.: 13/460,463

(22) Filed: Apr. 30, 2012

Related U.S. Application Data
(63) Continuation of application No. 11/081,802, filed on Mar. 17, 2005, now abandoned.
(60) Provisional application No. 60/553,554, filed on Mar. 17, 2004.
(60) Provisional application No. 60/584,674, filed on Jul. 2, 2004.

(51) Int. Cl. A61K 31/57; C07J 75/00; A61P 31/18; A61K 47/00; A61P 37/02; A61P 35/00; A61P 31/04; A61P 31/10; C07J 53/00; A61P 31/12

(52) U.S. Cl. 514/171; 552/510; 514/169; 424/278.1

(57) ABSTRACT

Salts of 3-O-(3′,3′-dimethylsuccinyl)Betulinic acid (DSB) are disclosed. Particularly, the preparation, pharmaceutical evaluation, and in vivo bioavailability evaluation of N-methyl-D-glucamine and alkali metal salt forms of DSB are disclosed. Pharmaceutical compositions including these salt forms are used in methods of treating HIV and related diseases. Methods of making the salts of DSB and the pharmaceutical compositions are also provided.

### Cross-Reference to Related Applications

This application is a continuation of U.S. application Ser. No. 11/081,802, filed Mar. 17, 2005, which claims the benefit of U.S. Provisional Application No. 60/553,554, filed Mar. 17, 2004 and U.S. Provisional Application No. 60/584,674, filed Jul. 2, 2004; the contents of all three of which are incorporated by reference herein in their entirety.

### BACKGROUND OF THE INVENTION

1. Field of the Invention

This invention relates to novel salt forms of 3-O-(3′,3′-dimethylsuccinyl) betulinic acid, also known as “DSB”. This invention also relates to methods of treating HIV and related diseases using pharmaceutical compositions comprising salt forms of DSB. The invention further relates to dosage forms of pharmaceutical compositions comprising salts of DSB.

2. Related Art

Human Immunodeficiency Virus (HIV) is a member of the lentiviruses, a subfamily of retroviruses. HIV infects and invades cells of the immune system; it breaks down the body's immune system and renders the patient susceptible to opportunistic infections and neoplasms. The immune defect appears to be progressive and irreversible, with a high mortality rate that approaches 100% over several years.

HIV-1 is trophic and cytopathic for T4 lymphocytes, cells of the immune system that express the cell surface differentiation antigen CD4, also known as OKT4, T4 and leu3. The viral tropism is due to the interactions between the viral envelope glycoprotein, gp120, and the cell-surface CD4 molecules (Dalgleish et al., Nature 312:763-767, 1984). These interactions, not only mediate the infection of susceptible cells by HIV, but are also HIV-1 is trophic and cytopathic for T4 lymphocytes, cells of the immune system that express the cell surface differentiation antigen CD4, also known as OKT4, T4 and leu3. The viral tropism is due to the interactions between the viral envelope glycoprotein, gp120, and the cell-surface CD4 molecules (Dalgleish et al., Nature 312:763-767, 1984). These interactions, not only mediate the infection of susceptible cells by HIV, but are also responsible for the virus-induced fusion of infected and uninfected T cells. This cell fusion results in the formation of giant multinucleated syncytia, cell death, and progressive depletion of CD4 cells in AIDS patients. These events result in HIV-induced immunosuppression and its subsequent sequelae, opportunistic infections and neoplasms.

In addition to CD4+ T cells, the host range of HIV includes cells of the mononuclear phagocytic lineage (Dalgleish et al., supra), including blood monocytes, tissue macrophages, Langerhans cells of the skin and dendritic reticulum cells within lymph nodes. HIV is also neurotropic, capable of infecting monocytes and macrophages in the central nervous system causing severe neurologic damage. Macrophage/monocytes are a major reservoir of HIV. They can interact and fuse with CD4-bearing T cells, causing T cell depletion and thus contributing to the pathogenesis of AIDS.

Considerable progress has been made in the development of drugs for HIV-1 therapy. Therapeutic agents for HIV can include, but not are not limited to, at least one of AZT, 3TC, ddC, d4T, ddI, tenofovir, abacavir, nevirapine, delavirdine, efavirenz, saquinavir, ritonavir, indinavir, nelfinavir, lopinavir, amprenavir and atazanavir, or any other antiretroviral drugs or antibodies in combination with each other, or associated with a biologically based therapeutic, such as, for example, gp41-derived peptides enfuvirtide (Fuzeon; Timeris-Roche), or soluble CD4, antibodies to CD4, and conjugates of CD4 or anti-CD4, or as additionally presented herein. Combinations of these drugs are particularly effective and can reduce levels of viral RNA to undetectable levels in the plasma and slow the development of viral resistance, with resulting improvements in patient health and life span.

Despite these advances, there are still problems with the currently available drug regimens. Many of the drugs exhibit severe toxicities, have other side-effects (e.g., fat redistribution) or require complicated dosing schedules that reduce compliance and thereby limit efficacy. Resistant strains of HIV often appear over extended periods of time even on combination therapy. The high cost of these drugs is also a limitation to their widespread use, especially outside of developed countries.

There is still a major need for the development of additional drugs to circumvent these issues. Ideally these would target different stages in the viral life cycle, adding to the armamentarium for combination therapy, and exhibit minimal toxicity, yet have lower manufacturing costs.

Betulinic acid and platanic acid have been isolated from Syzigium claviflorum and were determined to have anti-HIV activity. Betulinic acid and platanic acid exhibited inhibitory activity against HIV-1 replication in H9 lymphocyte cells with EC₅₀ values of 1.4 μM and 6.5 μM, respectively, and therapeutic index (T.I.) values of 9.3 and 14, respectively. Hydrogenation of betulinic acid yielded dihydrobetulinic acid, which showed slightly more potent anti-HIV activity with an EC₅₀ value of 0.9 and a T.I. value of 14 (Fujioka, T., et al., J. Nat. Prod. 57:243-247 (1994)). Esterification of betulinic acid with certain substituted acyl groups, such as 3′,3′-dimethylglutaryl and 3′,3′-dimethylsuccinyl groups produced derivatives having enhanced activity (Kashiwada, Y., et al., J. Med. Chem. 39:1016-1017 (1996)). Acylated betulinic acid and dihydrobetulinic acid derivatives that are potent anti-HIV agents are also described in U.S. Pat. No. 5,679,828. Anti-HIV assays indicated that 3-O-(3′,3′-dimethylsuccinyl)-betulinic acid and the dihydrobetulinic acid analog both demonstrated extremely potent anti-HIV activity in acutely infected H9 lymphocytes with EC₅₀ values of less than 1.7×10⁻⁵ μM, respectively. These compounds exhibited remarkable T.I. values of more than 970,000 and more than 400,000, respectively.

U.S. Pat. No. 5,468,888 discloses 28-amido derivatives of lupanes that are described as having a cytoprotecting effect for HIV-infected cells.

A number of triterpenoids, including betulinic acid, have several known medical applications, including use as an anticancer drug. Anderson et al., in WO 95/04526, discuss derivatives of triterpenoids which have been used in cancer therapy, including their activity against polyamines which are required by cells to grow at an optimal rate. Some of these triterpenoids have been found to interfere with the enzymatic synthesis of polyamines required for optimum cell growth, and thus inhibit the growth of cancer cells, particularly by inhibiting ornithine decarboxylase. Betulinic acid has been reported also to possess anti-inflammatory activity, which may be due to its capacity to inhibit enzymes involved in leukotriene biosynthesis, including 5-lipoxygenase.

Chan, in British patent 1,425,601, discloses that carboxylated derivatives of dihydrobetulinic acid can be included in pharmaceutical compositions, but there is no hint as to what use these compounds have.

Japanese Patent Application No. JP 01 143,832 discloses that betulin and 3,28-diesters thereof are useful in the anti-cancer field.

U.S. Pat. No. 6,172,110 B1, hereby incorporated herein by reference in its entirety, discloses betulin and dihydrobetulin derivatives which have the following formulae or pharmaceutically acceptable salts thereof,

### Betulin and Dihydrobetulin Derivatives

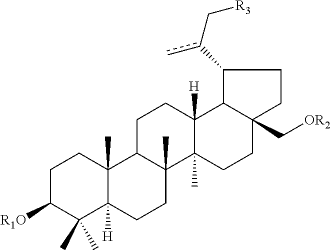

wherein R₁ is a C₂-C₂₀ substituted or unsubstituted carboxyacyl, R₂ is a C₂-C₂₀ substituted or unsubstituted carboxyacyl; and R₃ is hydrogen, halogen, amino, optionally substituted mono- or di-alkylamino, or —OR₄, where R₄ is hydrogen, C₁₋₄ alkanoyl, benzoyl, or C₂-C₂₀ substituted or unsubstituted carboxyacyl; wherein the dashed line represents an optional double bond between C₂₀ and C₂₉.

Derivatives in U.S. Pat. No. 6,172,110 B1 are formed by introducing a C₂ to C₂₀ substituted or unsubstituted acyl group at the C3-hydroxy or C28-hydroxy group of betulin and dihydrobetulin to produce the corresponding 3-O-acyl and/or 28-O-acyl derivatives. These compounds were described as useful for treating a subject infected with a retroviral infection, particularly HIV, by administering at least one of the aforementioned betulin derivatives optionally in combination with one or more known anti-AIDS therapeutic or immunostimulant.

U.S. Patent Application No. 60/413,451 discloses 3,3-dimethylsuccinyl betulin and is herein incorporated by reference. Zhu, Y-M. et al., Bioorg. Chem Lett. 11:3115-3118 (2001); Kashiwada Y. et al., J. Nat. Prod. 61:1090-1095 (1998); Kashiwada Y. et al., J. Nat. Prod. 63:1619-1622 (2000); and Kashiwada Y. et al., Chem. Pharm. Bull. 48:1387-1390 (2000) disclose dimethylsuccinyl betulinic acid and dimethylsuccinyl oleanolic acid. Esterification of the 3′ carbon of betulin with succinic acid produced a compound capable of inhibiting HIV-1 activity (Pokrovskii, A. G. et al., Gos. Nauchnyi Tsentr Virusol. Biotekhnol. “Vector,” 9:485-491 (2001)).

Published International Application No. WO 02/26761 discloses the use of betulin and analogs thereof for treating fungal infections.

There exists a need for new HIV inhibition methods that are effective against drug resistant strains of the virus. In one embodiment, the present invention provides therapeutic methods and compounds that inhibit the virus in different ways from approved therapies. In a particular embodiment, the present invention provides a therapeutic composition comprising a salt of DSB having enhanced solubility and bioavailability that inhibit the virus in vivo in different ways from approved therapies. The compositions of the present invention can be used to treat HIV-1 infection in human beings.

The compound and methods of the present invention have a novel mechanism of action and therefore are active against HIV strains that are resistant to current therapies. As such, this invention offers a completely new approach for treating HIV/AIDS.

### BRIEF SUMMARY OF THE INVENTION

The present invention relates to particular salt forms of 3-O-(3′,3′-dimethylsuccinyl) betulinic acid (“DSB”), their preparation, pharmaceutical compositions thereof, and methods of use thereof. Particularly, this invention relates to amine salts, such as the N-methyl-D-glucamine (NmG) salt form of DSB. This invention also relates to pharmaceutical compositions and dosage forms comprising these salt forms of DSB. These compositions and dosage forms can be used in methods of treating HIV and related diseases. Methods of making the salts of DSB and the pharmaceutical compositions are also provided.

### DETAILED DESCRIPTION OF THE INVENTION

A first aspect of the present invention is directed to a salt form of 3-O-(3′,3′-dimethylsuccinyl) betulinic acid, hereinafter ‘DSB’. DSB has the following formula:

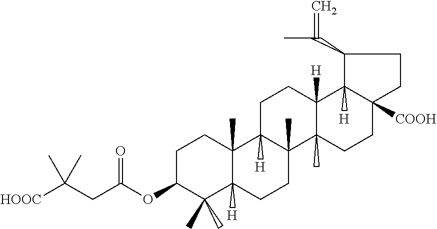

A second aspect of the present invention is drawn to an NmG salt of DSB. In one embodiment the glucamine salt of DSB is the di-(N-methyl-D-glucamine) salt of DSB (di-NmG salt). The di-(NmG) salt of DSB can have two NmG molecules bind per DSB molecule, has a molecular formula of C₅₀H₉₀N₂O₁₆, a molecular weight of 975.28 and has the following formula:

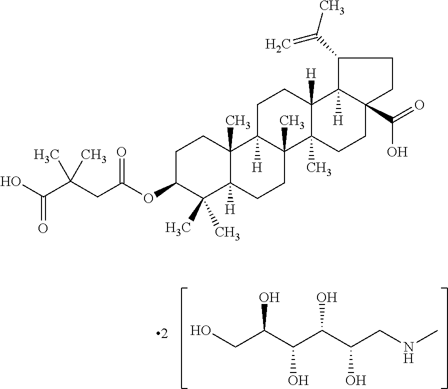

A third aspect of the present invention is drawn to a pharmaceutical composition comprising an NmG salt of DSB, such as the di-(N-methyl-D-glucamine) salt of DSB, and a pharmaceutical carrier or diluent.

A fourth aspect of the present invention is drawn to a method of preparing an NmG salt of DSB. In one embodiment of the invention, the method of preparing the salt comprises mixing NmG and DSB in an aqueous solution to provide 3-O-(3′,3′-dimethylsuccinyl)betulinic acid, N-methyl-D-glucamine salt. The mixing can occur in the presence of a cyclodextrin, such as hydroxypropyl-β-cyclodextrin.

A fifth aspect of the present invention is drawn to a dosage form, such as an oral tablet, comprising a pharmaceutical composition of an NmG salt of DSB. The dosage form can be used for treating, in a subject, a retroviral infection, such as HIV.

A sixth aspect of the present invention is drawn to a method of use of a pharmaceutical composition comprising an NmG salt of DSB for treating, in a human subject, a retroviral infection, such as HIV.

Any non-toxic, pharmaceutically acceptable amine or quaternary ammonium salt of DSB can be used in the present invention. These salts can be prepared in situ during the final isolation and purification of the compounds or by separately reacting the purified compound in its free acid form with a suitable organic base and isolating the salt thus formed. These include nontoxic ammonium, quaternary ammonium and amine cations including, but not limited to ammonium, tetra-methylammonium, tetraethylammonium, methylamine, dimethylamine, trimethylamine, ethylamine, N-methyl-glucamine and the like.

Of particular interest is the NmG salt form of DSB, namely 3-O-(3′,3′-dimethylsuccinyl)betulinic acid, N-methyl-D-glucamine salt and the alkali metal salt forms. These salt forms have been prepared by combining DSB with NmG or with an alkali metal hydroxide to provide mono- and di-salts of DSB. The salt forms of the present invention possess enhanced bioavailability.

The salts of the present invention have anti-retroviral activity, thus providing suitable compounds and compositions for treating retroviral infections, optionally with additional pharmaceutically active ingredients, such as anti-retroviral, anti-HIV, and/or immuno-stimulating compounds or antiviral antibodies or fragments thereof.

By the term “anti-retroviral activity” or “anti-HIV activity” is intended the ability to inhibit at least one of:

(1) viral pro-DNA integration into host cell genome;

(2) retroviral attachment to cells;

(3) viral entry into cells;

(4) cellular metabolism which permits viral replication;

(5) inhibition of intercellular spread of the virus;

(6) synthesis and/or cellular expression of viral antigens;

(7) viral budding or maturation;

(8) activity of virus-coded enzymes (such as reverse transcriptase, integrase and proteases); and/or

(9) any known retroviral or HIV pathogenic actions, such as, for example, immunosuppression. Thus, any activity which tends to inhibit any of these mechanisms is “anti-retroviral activity” or “anti-HIV activity.”

A salt of DSB of the present invention can be used for treatment of retroviral (e.g., HIV) infection either alone, or in combination with other modes of therapy known in the art. However, because the salts of DSB of the present invention are relatively less or substantially non-toxic to normal cells, their utility is not limited to the treatment of established retroviral infections. For example, a salt of DSB according to the present invention can be used in treating blood products, such as those maintained in blood banks The nation's blood supply is currently tested for antibodies to HIV. However, the test is still imperfect and samples which yield negative tests can still contain HIV virus. Treating the blood and blood products with the salts of DSB of the present invention can add an extra margin of safety by killing any retrovirus that may have gone undetected.

A salt of DSB according to the present invention can be used in the treatment of HIV in patients who are not adequately treated by other HIV-1 therapies. Accordingly, the invention is also drawn to a method of treating a patient in need of therapy, wherein the HIV-1 infecting said cells does not respond to other HIV-1 therapies. In another embodiment, methods of the invention are practiced on a subject infected with an HIV that is resistant to a drug used to treat HIV infection. In various applications, the HIV is resistant to one or more protease inhibitors, reverse transcriptase inhibitors, entry inhibitors, nucleoside analogs, vaccines, binding inhibitors, immunomodulators, and/or any other inhibitors. In some embodiments, the compositions and methods of the invention are practiced on a subject infected with an HIV that is resistant to one or more drugs used to treat HIV infections, for example, but not limited to, zidovudine, lamivudine, didanosine, zalcitabine, stavudine, abacavir, nevirapine, delavirdine, emtricitabine, efavirenz, saquinavir, ritonavir, lopinavir, indinavir, nelfinavir, tenofovir, amprenavir, adefovir, atazanavir, fosamprenavir, enfuvirtide, hydroxyurea, AL-721, ampligen, butylated hydroxytoluene; polymannoacetate, castanospermine; contracan; creme pharmatex, CS-87, penciclovir, famciclovir, acyclovir, cytofovir, ganciclovir, dextran sulfate, D-penicillamine trisodium phosphonoformate, fusidic acid, HPA-23, eflornithine, nonoxynol, pentamidine isethionate, peptide T, phenytoin, isoniazid, ribavirin, rifabutin, ansamycin, trimetrexate, SK-818, suramin, UA001, and combinations thereof.

In addition, a salt of DSB of the present invention can be used as a prophylactic to prevent transmission of HIV infection between individuals. For example, a salt of DSB can be administered orally or by injection to an HIV infected pregnant woman and/or fetus during pregnancy or immediately prior to, at, or subsequent to birth, to reduce the probability that the newborn infant becomes infected. Also, a salt of DSB can be administered vaginally immediately prior to childbirth to prevent infection of the infant during passage through the birth canal. Further, a salt of DSB of the present invention can be used during sexual intercourse to prevent transmission of HIV by applying a retroviral inhibiting effective amount of a topical composition including one or more salts of DSB to vaginal or other mucosa prior to sexual intercourse. For example, a salt of DSB of the present invention can be used to prevent transmission of HIV from an infected male to an uninfected female or vice versa.

Pharmaceutical compositions of the present invention can comprise at least one salt of DSB optionally in combination with one or more additional agent as described herein. Likewise, methods of treatment will employ pharmaceutical compositions that include at least one salt of DSB, as described herein, alone or in combination with additional agents as further described. Such modes of therapy can include chemotherapy with at least one additional drug as presented herein.

In one embodiment, a pharmaceutical composition according to the present invention can comprise at least one other anti-viral agents such as, but not limited to, AZT (zidovudine, RETROVIR®, GlaxoSmithKline), 3TC (lamivudine, EPIVIR®, GlaxoSmithKline), AZT+3TC, (COMBIVIR®, GlaxoSmithKline), AZT+3TC+abacavir (TRIZIVIR®, GlaxoSmithKline), ddI (didanosine, VIDEX®, Bristol-Myers Squibb), ddC (zalcitabine, HIVID®, Hoffmann-La Roche), D4T (stavudine, ZERIT®, Bristol-Myers Squibb), tenofovir (VIREAD®, Gilead), abacavir (ZIAGEN®, GlaxoSmithKline), nevirapine (VIRAMUNE®, Boehringer Ingelheim), delavirdine (Pfizer), efavirenz (SUSTIVA®, DuPont Pharmaceuticals), saquinavir (INVIRASE®, FORTOVASE®, Hoffmann-LaRoche), ritonavir (NORVIR®, Abbott Laboratories), indinavir (CRIXIVAN®, Merck and Company), nelfinavir (VIRACEPT®, Pfizer), lopinavir, amprenavir (AGENERASE®, GlaxoSmithKline), adefovir (PREVEON®, HEPSERA®, Gilead Sciences), atazanavir (Bristol-Myers Squibb), fosamprenavir (LEXIVA®, GlaxoSmithKline) and hydroxyurea (HYDREA®, Bristol-Meyers Squibb), or any other antiretroviral drugs or antibodies in combination with each other, or associated with a biologically based therapeutic, such as, for example, gp41-derived peptides enfuvirtide (FUZEON®, Roche and Trimeris) and T-1249, or soluble CD4, antibodies to CD4, and conjugates of CD4 or anti-CD4, or as additionally presented herein.

Additional suitable antiviral agents for optimal use with at least one salt of DSB can include, but are not limited to amphotericin B (FUNGIZONE®); Ampligen (mismatched RNA) developed by Hemispherx Biopharma; BETASERON® (β-interferon, Chiron); butylated hydroxytoluene; Carrosyn (polymannoacetate); Castanospermine; Contracan (stearic acid derivative); Creme Pharmatex (containing benzalkonium chloride); 5-unsubstituted derivative of zidovudine; penciclovir (DENAVIR® Novartis); famciclovir (FAMVIR® Novartis); acyclovir (ZOVIRAX® GlaxoSmithKline); cytofovir (VISTIDE® Gilead); ganciclovir (CYTOVENE®, HoffmanLaRoche); dextran sulfate; D-penicillamine (3-mercapto-D-valine); FOSCARNET® (trisodium phosphonoformate; AstraZeneca); fusidic acid; glycyrrhizin (a constituent of licorice root); HPA-23 (ammonium-21-tungsto-9-antimonate); ORNIDYL® (eflornithine; Aventis); nonoxynol; pentamidine isethionate (PENTAM-300); Peptide T (octapeptide sequence, Peninsula Laboratories); Phenytoin (Pfizer); INH or isoniazid; ribavirin (VIRAZOLE®, Valeant Pharmaceuticals); rifabutin, ansamycin (MYCOBUTIN® Pfizer); CD4-IgG2 (Progenics Pharmaceuticals) or other CD4-containing or CD4-based molecules; Trimetrexate (Medimmune); suramin and analogues thereof (Bayer); and WELLFERON® (α-interferon, GlaxoSmithKline).

Pharmaceutical compositions of the present invention can also further comprise immunomodulators. Suitable immunomodulators for optional use with a betulinic acid or betulin derivative of the present invention in accordance with the present invention can include, but are not limited to: ABPP (Bropririmine); Ampligen (mismatched RNA, Hemispherx Biopharma); anti-human interferon-α-antibody; ascorbic acid and derivatives thereof; interferon-β; Ciamexon; cyclosporin; cimetidine; CL-246,738; colony stimulating factors, including GM-CSF; dinitrochlorobenzene; HE2000 (Hollis-Eden Pharmaceuticals); inteferon-γ; glucan; hyperimmune gamma-globulin (Bayer); immuthiol (sodium diethylthiocarbamate); interleukin-1 (Hoffmann-LaRoche; Amgen), interleukin-2 (IL-2) (Chiron); isoprinosine (inosine pranobex); Krestin; LC-9018 (Yakult); lentinan (Yamanouchi); LF-1695; methionine-enkephalin; Minophagen C; muramyl tripeptide, MTP-PE; naltrexone (Barr Laboratories); RNA immunomodulator; REMUNE® (Immune Response Corporation); RETICULOSE® (Advanced Viral Research Corporation); shosaikoto; ginseng; thymic humoral factor; Thymopentin; thymosin factor 5; thymosin 1 (ZADAXIN®, SciClone); thymostimulin; TNF (tumor necrosis factor, Genentech); and vitamin preparations.

Pharmaceutical compositions of the present invention can also further comprise anti-cancer therapeutic agents. Suitable anti-cancer therapeutic agents for optional use include an anti-cancer composition effective to inhibit neoplasia comprising a compound, or a pharmaceutically acceptable salt or prodrug of said anti-cancer agent, which can be used for combination therapy include, but are not limited to, alkylating agents, such as busulfan, cis-platin, mitomycin C, and carboplatin antimitotic agents, such as colchicine, vinblastine, taxols, such as paclitaxel (TAXOL®, Bristol-Meyers Squibb) docetaxel (TAXOTERE®, Aventis), topo I inhibitors, such as camptothecin, irinotecan and topotecan (HYCAMTIN®, GlaxoSmithKline), topo II inhibitors, such as doxorubicin, daunorubicin and etoposides such as VP16; RNA/DNA antimetabolites, such as 5-azacytidine, 5-fluorouracil and methotrexate, DNA antimetabolites, such as 5-fluoro-2′-deoxy-uridine, ara-C, hydroxyurea, thioguanine, and antibodies, such as trastuzumab (HERCEPTIN®, Genentech), and rituximab (RITUXAN®, Genentech and Biogen-Idec), melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, alanosine, and combinations thereof.

The invention further provides methods for providing anti-bacterial therapeutics, anti-parasitic therapeutics, and anti-fungal therapeutics, for use in combination with the compounds of the invention and pharmaceutically-acceptable salts thereof. Examples of anti-bacterial therapeutics include compounds such as penicillins, ampicillin, amoxicillin, cyclacillin, epicillin, methicillin, nafcillin, oxacillin, cloxacillin, dicloxacillin, flucloxacillin, carbenicillin, cephalexin, cepharadine, cefadoxil, cefaclor, cefoxitin, cefotaxime, ceftizoxime, cefinenoxine, ceftriaxone, moxalactam, imipenem, clavulanate, timentin, sulbactam, erythromycin, neomycin, gentamycin, streptomycin, metronidazole, chloramphenicol, clindamycin, lincomycin, quinolones, rifampin, sulfonamides, bacitracin, polymyxin B, vancomycin, doxycycline, methacycline, minocycline, tetracycline, amphotericin B, cycloserine, ciprofloxacin, norfloxacin, isoniazid, ethambutol, and nalidixic acid, as well as derivatives and altered forms of each of these compounds.

Examples of anti-parasitic therapeutics include bithionol, diethylcarbamazine citrate, mebendazole, metrifonate, niclosamine, niridazole, oxamniquine and other quinine derivatives, piperazine citrate, praziquantel, pyrantel pamoate and thiabendazole, as well as derivatives and altered forms of each of these compounds.

Examples of anti-fungal therapeutics include amphotericin B, clotrimazole, econazole nitrate, flucytosine, griseofulvin, ketoconazole and miconazole, as well as derivatives and altered forms of each of these compounds. Anti-fungal compounds also include aculeacin A and papulocandin B.

A preferred animal subject of the present invention is a human being. In a particular embodiment, the present invention is useful in the treatment of human patients.

The term “treating” means the administering to subjects a salt of DSB according to the present invention for purposes which can include prevention, amelioration, or cure of a retroviral-related pathology.

Medicaments are considered to be provided “in combination” with one another if they are provided to the patient concurrently or if the time between the administration of each medicament is such as to permit an overlap of biological activity.

In one embodiment of the present invention, a pharmaceutical composition comprises the di-(N-methyl-D-glucamine) salt of 3-O-(3′,3′-dimethylsuccinyl)betulinic acid.

Pharmaceutical compositions for administration according to the present invention comprising at least one salt of DSB according to the present invention in a pharmaceutically acceptable form are optionally combined with a pharmaceutically acceptable carrier. These compositions can be administered by any means that achieve their intended purposes. Amounts and regimens for the administration of the salts of DSB according to the present invention can be determined readily by those with ordinary skill in the clinical art of treating a retroviral pathology.

For example, administration can be by parenteral, such as subcutaneous, intravenous, intramuscular, intraperitoneal, transdermal, or buccal routes. Alternatively, or concurrently, administration can be by the oral route. The dosage administered depends upon the age, health and weight of the recipient, type of previous or concurrent treatment, if any, frequency of treatment, and the nature of the effect desired.

Compositions within the scope of this invention include all compositions comprising at least one salt of DSB according to the present invention in an amount effective to achieve its intended purpose. While individual needs vary, determination of optimal ranges of effective amounts of each component is within the skill of the art. Typical dosages of at least one salt of DSB comprise about 0.05 to about 100 mg/kg body weight. In some embodiments, a useful dosage of one or more salts of DSB comprises about 0.1 to about 100 mg/kg body weight of the active ingredient, preferably about 0.1 to about 20 mg/kg body weight of the active ingredient. In some embodiments, a more preferred dosage of one or more salts of DSB comprises about 0.2 to about 10 mg/kg body weight. A useful dosage of one or more salts of DSB comprises about 0.5 to about 5 mg/kg body weight. In some embodiments, the dosage of one or more salts of DSB can comprise about 10 to about 100 mg/kg body weight.

Various dosage amounts of the composition of the invention can be administered to provide various plasma levels of DSB. In some embodiments, a preferred dosage amount is one which provides a trough concentration of DSB in the patient's plasma of about 1 micromolar (μM) to about 1 millimolar (mM). In some embodiments, the dosage amount is one which provides a trough concentration of DSB in the patient's plasma of about 4 μM (2.34 μg/mL) to about 1000 μM, about 40 μM to about 1000 μM, or about 400 μM to about 1000 μM. In some embodiments, the dosage amount is one which provides a trough concentration of DSB in the patient's plasma of about 4 μM (2.34 μg/mL) to about 200 μM, about 10 μM to about 200 μM, or about 40 μM to about 200 μM. In some embodiments, the dosage amount is one which provides a trough concentration of DSB in the patient's plasma of at least about 4 μM (2.34 μg/mL) or greater, at least about 10 μM or greater, at least about 40 μM or greater, at least about 100 μM or greater, or at least 200 μM or greater. In some embodiments, the dosage amount is one which provides a trough concentration of DSB in the patient's plasma of about 400 μM. The “trough concentration” is the concentration of DSB in the patient's plasma just prior to subsequent dosing of the patient.

Various amounts of one or more salts of the present invention can be administered according to the present invention. In some embodiments, about 10 mg to about 1000 mg of the active ingredients of one or more salts of the present invention can be administered once per day. In some embodiments, about 50 mg to about 500 mg of the active ingredient of one or more salts of the present invention can be administered once per day. In some embodiments, 25 mg, 50 mg, 75 mg, 100 mg, 150 mg, 200 mg, 250 mg, 300 mg, or 500 mg of the active ingredient of one or more salts of the present invention is administered once per day. The amount of one or more salts administered per day is determined by the total amount of one or more salts administered in a 24 hour period. Thus, dosage regimens which instruct administration of one or more salts of the invention multiple times during a 24 hour period are within the scope of the invention if the cumulative amount administered during a 24 hour period is within the ranges listed above.

Therapeutic administration can also include prior, concurrent, subsequent or adjunctive administration of at least one additional salt of DSB according to the present invention or other therapeutic agent, such as an anti-viral or immune stimulating agent. In such an approach, the dosage of the second drug can be the same as or different from the dosage of the first therapeutic agent. In one embodiment of the present invention, the drugs are administered on alternate days in the recommended amounts of each drug.

Administration of a compound of the present invention can also optionally include previous, concurrent, subsequent or adjunctive therapy using immune system boosters or immunomodulators. In addition to the pharmacologically active compounds, a pharmaceutical composition of the present invention can also contain suitable pharmaceutically acceptable carriers comprising excipients and auxiliaries which facilitate processing of the active compounds into preparations which can be used pharmaceutically. In one embodiment, the preparations, particularly those preparations which can be administered orally, such as tablets, dragees, and capsules, and also preparations which can be administered rectally, such as suppositories, as well as suitable solutions for administration by injection or orally, contain from about 0.01 to 99 percent of the active ingredient together with the excipient. In another embodiment, the preparation can include from about 20 to 75 percent of active compound(s), together with the excipient.

Pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture, and processing the mixture of granules, after adding suitable auxiliaries, if desired or necessary, to obtain tablets or dragee cores.

Suitable excipients are, e.g., fillers such as saccharide, for example, lactose or sucrose, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl cellulose phthalate are used. Dyestuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds can be dissolved or suspended in suitable liquids, such as fatty oils or liquid paraffin. In addition, stabilizers can be added.

Possible pharmaceutical preparations which can be used rectally include, for example, suppositories which consist of a combination of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts. In addition, suspensions of the active compounds as appropriate oily injection suspensions can be administered. Suitable lipophilic solvents or vehicles include fatty oils, such as sesame oil, or synthetic fatty acid esters, such as ethyl oleate or triglycerides. Aqueous injection suspensions that can contain substances which increase the viscosity of the suspension include, for example, sodium carboxymethyl cellulose, sorbitol, and/or dextran. Optionally, the suspension can also contain stabilizers.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as, for example, water or other solvents, solubilizing agents and emulsifiers such as ethyl alcohol, isopropyl alcohol, cyclodextrins such as hydroxypropyl-β-cyclodextrin, ethyl carbonate, ethyl acetate, benzyl alcohol, benzyl benzoate, propylene glycol, 1,3-butylene glycol, dimethyl formamide, oils such as cottonseed, groundnut, corn, germ, olive, castor, and sesame oils, glycerol, tetrahydrofurfuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, cellulose, microcrystalline cellulose, aluminum metahydroxide, bentonite, agar-agar, and tragacanth, and combinations thereof.

A pharmaceutical formulation for systemic administration according to the invention can be formulated for enteral, parenteral or topical administration. Indeed, all three types of formulation can be used simultaneously to achieve systemic administration of the active ingredient.

Suitable formulations for oral administration include oral dosage forms such as, but not limited to, hard or soft gelatin capsules, dragees, pills, tablets, including coated tables, elixirs, suspensions, syrups or inhalations and controlled release forms thereof.

Solid dosage forms in addition to those formulated for oral administration include rectal suppositories.

The salts of DSB of the present invention can also be administered in the form of an implant when compounded with a biodegradable slow-release carrier. Alternatively, the salts of DSB of the present invention can be formulated as a transdermal patch for continuous release of the active ingredient.

Suitable formulations for topical administration include creams, gels, jellies, mucilages, pastes and ointments. Suitable injectable solutions include intravenous subcutaneous and intramuscular injectable solutions. Alternatively, the salts of DSB can be administered in the form of an infusion solution, a nasal inhalation or spray, or a mucosal or vaginal delivery system, such as a vaginal ring, foam, cream, gel, medicated suppository and medicated tampon.

Prophylactic topical compositions for preventing HIV infection between individuals during childbirth or sexual intercourse include one or more salts of DSB and at least one pharmaceutically acceptable topical carrier or diluent. The topical composition can be, for example, in the form of an ointment, a cream, a gel, a lotion, a paste, a jelly, a spray, a foam, or a sponge. The dosage amount of a salt of DSB in a prophylactic topical formulation is, in general, less than about 1,000 milligrams, and in some embodiments between about 0.01 to about 100 milligrams. The topical formulations can include other prophylactic ingredients. The carrier and diluents should be acceptable in the sense of being compatible with other ingredients of the formulation and not deleterious to the recipient.

Topical prophylactic formulations include those suitable for vaginal, rectal or topical administration. The formulations can, where appropriate, be conveniently presented in discrete dosage units, and can be prepared by any of the methods known in the art of pharmacy. All such methods include the step of bringing the active agent into association with liquid carriers, gels or finely divided solid carriers or both and then, if necessary, shaping the product into the desired formulation.

Prophylactic formulations suitable for vaginal administration can be presented as pessaries, tampons, creams, gels, pastes, jelly, foams, or sprays, or aqueous or oily suspensions, solutions or emulsions (liquid formulations) containing suitable carriers known in the art in addition to the active agent. Liquid formulations can contain conventional additives, such as, suspending agents, emulsifying agents, non-aqueous vehicles including edible oils, or preservatives. These formulations are useful to prevent both sexual transmission of HIV and infection of an infant during passage through the birth canal. In one example, the vaginal administration can take place prior to sexual intercourse, or immediately prior to childbirth.

In some embodiments, prophylactic formulations suitable for rectal or vaginal administration having a solid carrier are represented as unit dose suppositories. Suitable carriers include cocoa butter and other materials commonly used in the art. Suppositories can be formed, for example, mixing one or more salts of DSB with one or more softened or melted carriers followed by chilling and shaping in molds.

Prophylactic formulations according to the invention can also be in the form of drops formulated with an aqueous or non-aqueous base comprising one or more dispersing agents, solubilizing agents, or suspending agents. Liquid sprays can be delivered from pressurized packs.

Prophylactic formulations according to the invention can be adapted to give sustained delivery. Also, the prophylactic formulations can include other active agents, such as spermicidal agents, antimicrobial agents, and anti-viral agents.

The triterpene derivatives of the present invention can also be administered in the form of an implant when compounded with a biodegradable slow-release carrier. Alternatively, the triterpene derivatives of the present invention can be formulated as a transdermal patch for continuous release of the active ingredient.

Salts of the present invention are made by mixing a basic or cation-forming compound, such as NmG, and DSB in an aqueous solution. The mixing can occur in the presence of a cyclodextrin, such as hydroxypropyl-β-cyclodextrin. The free acid of DSB can be obtained by the synthesis method described in U.S. Pat. No. 5,679,828.

Having now generally described the invention, the same will be more readily understood through reference to the following examples.

### EXAMPLE 1

### Preparation of di-(N-methyl-D-glucamine) salt of 3-O-(3′,3′-dimethylsuccinyl) betulinic acid

2.09740 g N-methyl-D-glucamine was dissolved in 250 mL methyl alcohol. 3.13295 g DSB was added and while sitting overnight, the suspension became a clear. The solvent was removed with a nitrogen gas stream. A thick, colorless oil formed. 200 mL methyl alcohol was added to dissolve the oil. Slow addition of 200 mL diethyl ether to the swirling mixture afforded a white solid. Isolation of the solid material by vacuum filtration afforded 5.51993 g crystalline solids. Drying of the solids for 72 hours under vacuum afforded 4.9737 g material.

### EXAMPLE 2

### Preparation of di-sodium salt of 3-O-(3′,3′-dimethylsuccinyl)betulinic acid

1.35531 g of DSB was dissolved in 50 mL methanol. 0.18758 g of solid sodium hydroxide was dissolved in 2.0 mL deionized water. The two mixtures were combined and diluted with an additional 15 mL methanol. After the mixture became clear, the methanol mixture was concentrated to 25 mL with a stream of nitrogen. 90 mL of diethyl ether was added. Vacuum filtration followed by vacuum drying at ambient temperatures yielded 1.45986 g of the disodium salt.

### EXAMPLE 3

### Preparation of di-potassium salt of 3-O-(3′,3′-dimethylsuccinyl) betulinic acid

4.14657 g of DSB was dissolved in 50 mL methanol. 0.95287 g of solid 85% potassium hydroxide was dissolved in 10 mL deionized water. The two mixtures were combined and diluted with an additional 250 mL methanol. After the mixture became clear, the methanol was removed with a stream of nitrogen. The resulting white solid was dissolved in 50 mL methanol and precipitated with 200 mL diethyl ether. Vacuum filtration followed by vacuum drying at ambient temperatures yielded 4.29679 g of the dipotassium salt.

### EXAMPLE 4

### Evaluation of Salts of the Present Invention and Comparative Examples

### A. Solubility

Qualitative results on the solubilities of the NmG, potassium and sodium salt forms of DSB (respectively, DSB-(NmG)₂, DSB-K₂ and DSB-(Na)₂) are presented in Tables 1, 2 and 3 respectively. Attempted concentrations are reported in mg/mL, except where the excipients were added by weight. These concentrations are reported in mg/g. The listed concentrations do not take into account correction factors for purity or additional molar mass due to the salt. In most cases, a successful formulation is described as a clear solution. However, some formulations were slightly tinted or exhibited haziness even when the drug substance was solubilized. Most of the formulations were mixed at room temperature on a magnetic stir plate. Formulations containing Vitamin E TPGS (di-α-tocopheryl polyethylene glycol 1000 succinate) were maintained at 40-45° C. in order to keep the mixture in a liquid state.

Of particular interest is the solubility of the three salt forms in a solvent consisting of 10% hydroxypropyl-β-cyclodextrin in water. Whereas an attempted solution of 27.7 mg/mL of DSB-(Na)₂ in this solvent system is cloudy, a solution of 50.0 mg/mL of DSB-(NmG)₂ in this solvent is clear. This suggests a significantly higher solubility of DSB-(NmG)₂ in this solvent than DSB-(Na)₂.

The solubility of the DSB-(NmG)₂ as a function of pH was measured at 25° C. DSB-(NmG)₂ was visibly soluble in basic and neutral conditions. A solubility of 7.537 mg/mL was observed at a pH of 9.461, and a solubility of 7.463 mg/mL was observed at a pH of 10.691. DSB-(NmG)₂ was visually insoluble in strongly acid solutions at 25° C.

The equilibrium concentration of DSB-(NmG)₂ is very high in deionized water and propylene glycol, both highly polar solvents (see Table 4). Comparing the solubility of DSB-(NmG)₂ in deionized water (>18 mg/mL, dielectric constant=88.0), in methanol (37.03 mg/mL, dielectric constant=32.6), and in ethanol (24.99 mg/mL, dielectric constant=24.3), it is evident that the solubility decreases with dielectric constant. This is consistent with the ionic nature of DSB-(NmG)₂.

TABLE 1
DSB—(NmG)₂

| | | Attempted | |
|---|---|---|---|
| Solvent System | | Concentration, | |
| (%) | | (mg/mL) | Appearance |
| Water | 100 | 2.4 | Solution cloudy |
| Captex 200 | 100 | 24.7 mg/g | Solution cloudy |
| Hydroxypropyl- | 10 | 50.0 | Solution clear, but foam |
| β-cyclodextrin | | | present |
| (in water) | | | |
| Water | 56 | 50.9 | Very foamy, drug is |
| PEG 400^{†} | 40 | | apparently undissolved |
| Propylene Glycol | 4 | | |
| Water | 67 | 50.8 | Solution clear, initially |
| PEG 400 | 29 | | foamy which eventually |
| Ethanol | 4 | | dissipates |
| Water | 78 | 51.2 | Solution clear, initially |
| PEG 400 | 18 | | foamy which eventually |
| Ethanol | 4 | | dissipates |
| Water | 89 | 50.1 | Solution clear, initially |
| PEG 400 | 7 | | foamy which eventually |
| Ethanol | 4 | | dissipates |
| Water | 83 | 51.7 | Solution clear, initially |
| PEG 400 | 7 | | foamy which eventually |
| Propylene Glycol | 6 | | dissipates |
| Ethanol | 4 | | |
| Water | 96 | 50.1 | Solution clear, initially |
| Ethanol | 4 | | foamy which eventually |
| | | | dissipates |
| Water | 75 | 50.9 | Undissolved drug, very |
| PEG 400 | 13 | | foamy, very viscous, |
| Vitamin E TPGS* | 10 | | gelling observed |
| Ethanol | 2 | | |
| Water | 75 | 50.1 | Solution clear, very |
| PEG 400 | 12 | | foamy, gelling |
| Vitamin E TPGS | 10 | | observed. |
| Ethanol | 3 | | |
| Water | 75 | 50.8 | Solution clear, very |
| PEG 400 | 11 | | foamy, gelling |
| Vitamin E TPGS | 10 | | observed. |
| Ethanol | 4 | | |
| Water | 84 | 50.5 | Solution cloudy, some |
| PEG 400 | 11 | | foam present |
| Ethanol | 4 | | |
| Simethicone | 1 | | |
| Emulsion | | | |
| Captex 200 | 81 | 25.5 | Solution cloudy, some |
| Vitamin E TPGS | 15 | | gelling present |
| DMA** | 4 | | |
| Water | 96 | 52.2 | Solutions clears with |
| DMA | 4 | | heating |
| Water | 96 | 71.2 | Solution cloudy |
| DMA | 4 | | |
| Captex 200 | 82 | 24.5 mg/g | Solution cloudy, some |
| Vitamin E TPGS | 16 | | gelling present |
| DMA | 2 | | |
| Water | 86 | 67.3 | Solution clear |
| PEG 400 | 10 | | |
| Ethanol | 4 | | |
| Hydroxypropyl- | 10 | 50.0 | Solution clear |
| β-cyclodextrin | | | |
| (in water) | | | |
| Water | 84 | 40.4 mg/g | Drug appears to be |
| PEG 400 | 10 | | dissolved, but solution |
| Ethanol | 4 | | is hazy from liquid |
| Vitamin E TPGS | 2 | | microscopic ‘structure’ |

^{†}Polyethyleneglycol 400;
*di-α-tocopheryl polyethylene glycol 1000 succinate;
**dimethylacetamide

TABLE 2
DSB—K₂

| | | Attempted | |
|---|---|---|---|
| Solvent System | | Concentration, | |
| (%) | | (mg/mL) | Appearance |
| Water | 100 | 3.5 | Solution cloudy |
| Water | 84 | 42.2 | Solution cloudy |
| PEG 400 | 12 | | |
| Ethanol | 4 | | |
| Hydroxypropyl-β- | 10 | 29.0 | Solution clear |
| cyclodextrin | | | |
| (in water) | | | |

TABLE 3
DSB—(Na)₂

| | | Attempted | |
|---|---|---|---|
| Solvent System | | Concentration, | |
| (%) | | (mg/mL) | Appearance |
| Hydroxypropyl-β- | 10 | 15.1 | Solution clear |
| cyclodextrin | | | |
| (in water) | | | |
| Hydroxypropyl-β- | 10 | 27.7 | Solution cloudy |
| cyclodextrin | | | |
| (in water) | | | |
| Hydroxypropyl-β- | 20 | 27.9 | Solution clear |
| cyclodextrin | | | |
| (in water) | | | |

TABLE 4
EQUILIBRIUM SOLUBILITY VALUES OF DSB—(NmG)₂ AND
DSB (FREE ACID) IN SELECTED SOLVENTS

| | Solubility of | | Solubility of DSB | Equilibration |
|---|---|---|---|---|
| Vehicle | DSB—(NmG)₂ | | Free Acid | Time (Days) |
| HERG Buffer | 17.76 | μg/mL | N/A | 3 |
| 1% DMSO in | 87.11 | μg/mL | N/A | 3 |
| HERG Buffer | | | | |
| Propylene glycol^{a} | >255 | mg/mL | N/A | 7 |
| PEG 400^{b} | 5.12 | mg/mL | N/A | 7 |
| Methanol | 37.03 | mg/mL | N/A | 7 |
| Ethanol^{c} | 24.99 | mg/mL | N/A | 7 |
| Deionized Water | >7 | mg/mL | N/A | 4 |
| PSS* | 80.18 | μg/mL | 1.50 mg/mL | 3 |
| PSS + 1% DMSO | 107.9 | μg/mL | 1.63 mg/mL | 3 |
| PSS + 0.1% DMSO | 107.7 | μg/mL | N/A | 3 |
| Deionized Water | >18 | mg/mL | 35.1 μg/mL | 3 |

^{a}Straw/amber solution color. All other samples were colorless or white.
^{b}Compound may have precipitated out of solution upon removal from the water bath, decreasing final concentration.
^{c} Upon transferring sample from equilibration vial to centrifuge tube, solution became cloudy. Solid may have precipitated out of solution. Initial visual assessment post equilibration was near unsaturated. Unsaturated concentration ~33 mg/mL. May be a result of temperature difference from equilibration bath and room temperature.
*Physiological salt solution
N/A = Not applicable due to samples not being run with these solvents.

### B. In Vivo Bioavailability

An oral bioavailability study was conducted in male, Sprague Dawley rats. The objective of this study was to compare the pharmacokinetics of various formulations of DSB administered as the free acid or as one of the salt forms: NmG, Na, or K. A solution or suspension of the free acid or salt was administered by oral gavage using a feeding needle to provide a nominal oral dose of 25 mg/kg. For each salt administered, the dose administered was based on free acid equivalents. The group number and formulations are listed below.

Group Number and Formulation
Group 1: 25 mg/mL DSB free acid in 2% DMA (dimethylacetamide), 14% Vitamin E TPGS, 84% Captex (a diester formed from selected high purity vegetable fatty acids and propylene glycol (triglyceride))
Group 2: 25 mg/mL of the DSB free acid in 4% Vitamin E TPGS and 96% carboxymethylcellulose (CMC) (0.5%) suspension in water.
Group 3: 41.7 mg/mL of DSB-(NmG)₂ salt in 84% water, 4% ethanol, and 12% PEG 400 (equivalent to 25 mg/mL of free acid)
Group 4: 41.7 mg/mL of the DSB-(NmG)₂ salt in 84% water, 4% ethanol, and 10% PEG 400, 2% Vitamin E TPGS (equivalent to 25 mg/mL of free acid)
Group 5: 41.7 mg/mL of the DSB-(NmG)₂ salt in 10% hydroxy-β-cyclodextrin in water (equivalent to 25 mg/mL of free acid)
Group 6: 25 mg/mL of the micronized DSB free acid in 0.5% CMC suspension
Group 7: 25 mg/mL of the micronized DSB free acid in 0.5% CMC suspension and Vitamin E TPGS
Group 8: 28.3 mg/mL of the DSB-K₂ salt in 10% hydroxy-β-cyclodextrin in water (equivalent to 25 mg/mL of free acid)
Group 9: 13.5 mg/mL of the DSB-(Na)₂ salt in 10% hydroxy-β-cyclodextrin in water (equivalent to 12.5 mg/mL of free acid)
Group 10: 25 mg/mL DSB free acid in 96% Captex 200, 4% Vitamin E TPGS

Blood samples were collected by cardiac puncture (n=2 rats/formulation/time point) at the following times: 0.25, 0.5, 1, 2, 4, 6, 12, and 24 h. Bioavailabilities of the oral formulations were calculated as the ratio of the oral dose-adjusted AUC_{INF} assuming proportionality of the dose adjustments to the dose adjusted AUC_{INF} reported after intravenous administration (AUC_{INF}=63.3 μg h/mL) from a study of DSB in rats.

The results of this study are presented in Table 5. The results of this study are presented in Table 5. The NmG salt formulations had the highest oral bioavailability, ranging from 49%-71%. The data for Groups 5, 8 and 9 show that, at equivalent doses, the rank order of bioavailability of the salts is NmG>potassium>sodium in a solvent of 10% hydroxypropyl-β-cyclodextrin in water.

TABLE 5
PHARMACOKINETIC PARAMETER VALUES FOR DSB IN PLASMA OF
RATS GIVEN SINGLE ORAL DOSES OF 25 MG/KG

| | Calculated | | | | | | |
|---|---|---|---|---|---|---|---|
| Group | Dose | Cₘₐₓ | tam | AUCₐₗₗ | AUC_{INF} | Apparent | F |
| Number | (mg/kg)^{a} | (μg/mL)^{b} | (h)^{c} | (μg · h/mL)^{d} | (μg · h/mL)^{e} | kₑ (h⁻¹)^{f} | (%)^{g} |
| 1 | 26.05 | 5.52 | 1.0 | 16.6 | 16.8 | 0.3327 | 10.6 |
| | | | | (0-12 h) | | | |
| 2 | 22.45 | 0.419 | 2.0 | 1.36 | 2.26 | 0.2051 | 1.65 |
| | | | | (0-6 h) | | | |
| 3 | 20.12 | 12.0 | 4.0 | 65.3 | 66.4 | 0.4675 | 54.2 |
| | | | | (0-12 h) | | | |
| 4 | 20.48 | 13.7 | 4.0 | 88.9 | 89.4 | 0.6019 | 71.7 |
| | | | | (0-12 h) | | | |
| 5 | 18.15 | 11.4 | 4.0 | 54.3 | 54.5 | 0.6496 | 49.3 |
| | | | | (0-12 h) | | | |
| 6 | 21.08 | 0.570 | 4.0 | 3.28 | 3.82 | 0.0837 | 2.98 |
| | | | | (0-24 h) | | | |
| 7 | 21.80 | 2.00 | 6.0 | 10.2 | 10.2 | 0.7486 | 7.69 |
| | | | | (0-12 h) | | | |
| 8 | 19.87 | 7.28 | 4.0 | 44.1 | 44.7 | 0.1971 | 37.0 |
| | | | | (0-24 h) | | | |
| 9 | 20.22 | 11.0 | 4.0 | 39.1 | 39.2 | 0.5496 | 31.9 |
| | | | | (0-12 h) | | | |
| 10 | 24.54 | 1.32 | 2.0 | 6.05 | 6.63 | 0.0876 | 6.70 |
| | | | | (0-12 h) | | | |

^{a}Measured concentration from dose solution analysis
^{b}Peak mean measured plasma concentration.
^{c}Time to peak concentration
^{d}Area under plasma concentration vs. time curve
^{e}Area under plasma concentration vs. time curve extrapolated to infinity
^{f}Elimination rate constant; half-life = 0.693/kₑ
^{g}Bioavailability was calculated as the dose-adjusted ratio of AUC_{INF} obtained after oral versus intravenous administration of DSB (dose = 10.4 mg/kg, AUC_{INF} = 63.3 mg · h/mL). Intravenous data were obtained from a study of DSB in rats.
F (%) = 100 × (Doseᵢₙₜᵣₐᵥₑₙₒᵤₛ × AUC_{INForal})/(Doseₒᵣₐₗ × AUC_{INFintravenous})

### EXAMPLE 5

### Characterization of the In Vivo Anti-HIV Efficacy of DSB-(NmG)₂ in the SCID-hu Thy/Liv Mouse Model of HIV Infection

This study was conducted to evaluate the antiviral activity of the NmG salt of DSB (DSB-(NmG)₂) at doses of 100 and 300 mg/kg/day in male SCID-hu Thy/Liv mice (7/group) treated twice daily by oral gavage for 22 days. (Dosages refer to free acid equivalents, i.e., the amount of DSB present in the dose.) At 300 mg/kg/day dose level, a group of uninfected SCID-hu Thy/Liv mice were dosed to assess the tolerability of this dose level. Once daily oral gavage of 3TC (lamivudine) at 30 mg/kg/day for 22 days was used as a positive control. Male C.B-17 SCID mice were obtained at 6 weeks of age and implanted with human fetal tissue from a single donor. At 14 weeks, mice were implanted with fragments of human fetal liver and human fetal thymus under the mouse kidney capsule to create the SCID-hu Thy/Liv mice. When the implants reached approximately 30 mm³ the animals were entered into the experiment. Inoculations of SCID-hu Thy/Liv mice with HIV-1 were performed on anesthetized mice in a restricted animal barrier facility under BSL3 guidelines. Each Thy/Liv implant was injected with 50 μL (1,000 TCID50) of NL4-3 batch JK WS1 D3 (diluted 1:2) or with RPMI 1640 medium in 1 to 3 places with a 250-μL Hamilton glass syringe and 30-gauge×½-inch blunt needle. For this study, implants were inoculated 18 weeks after tissue implantation. All implants were collected 21 days after inoculation.

The dosing vehicle was 10% hydroxypropyl-β-cyclodextrin in sterile PBS (phosphate buffered saline). Dosing solutions were prepared fresh before each administration at concentrations of 12 and 38 mg/mL for dosing at 100 and 300 mg/kg/day of DSB free acid.

Mice were dosed orally by gavage (200 μL/dose) twice per day and observed for signs of toxicity. Because toxicity was observed in mice treated at 300 mg/kg/day, the dosage level was reduced to 150 mg/kg/day beginning 7 days after treatment initiation after recovery period of 5 days without treatment. Treated mice were observed at the time dosing for signs of toxicity and were weighed every 2-5 days. Mice were inoculated with NL4-3 one day after the first dose (1-2 hr after the AM dose), and dosing was performed for 22 days.

Twenty-one days after inoculation, the Thy/Liv implants were surgically excised and transferred into 6-well tissue culture plates containing cold sterile PBS/2% FBS (fetal bovine serum). A single cell suspension was made by placing the implant into a sterile nylon mesh bag, submerging the bag in PBS/2% FBS in a 60-mm tissue culture dish, and dispersing the tissue between the nylon layers with forceps. The cells were counted with a Coulter counter, and appropriate numbers of cells were aliquoted for each assay. For p24 ELISA, pellets of 2.5×10⁶ cells were resuspended in 400 μL of p24 lysis buffer, rotated overnight at 4° C., and stored at −20° C. For RNA quantitation by branched DNA assay, dry pellets of 5×10⁶ cells were frozen and stored at −80° C. For FACS analysis, 10⁶ cells per well were placed in a 96-well plate for fixation and staining and were analyzed on the same day by four-color FACS analysis.

Twice-daily oral gavage of DSB-(NmG)₂ at 100 and 150 mg/kg/day beginning 24 hr before virus inoculation had potent, dose-dependent antiviral activity against NL4-3 in this study. At the highest dosage level, p24 was reduced by 99% (14 versus 440 pg p24 per 10⁶ cells for untreated mice) and HIV-1 RNA by 97% (10^{3.4} versus 10^{5.6} copies per 10⁶ cells), and MHC-I expression on CD4⁺CD8⁺ thymocytes was reduced to more normal levels (250 versus 530 in mean fluorescence intensity). There were modest, yet statistically significant reductions in implant p24 and viral RNA at 100 mg/kg/day (69 pg p24 and 10^{4.0} copies per 10⁶ cells), and there were no reductions after treatment with the 10% hydroxypropyl-β-cyclodextrin vehicle alone (590 pg 24 and 10^{5.6} copies per 10⁶ cells).

Once-daily oral gavage of 3TC at 100 mg/kg/day beginning 24 hr before virus inoculation had demonstrable antiviral activity in this study, reducing p24 by 97% (35 versus 440 pg p24 per 10⁶ cells) and HIV-1 RNA by 96% (10^{4.2} versus 10^{5.6} copies per 10⁶ cells) and preserving thymocyte viability (71% live thymocytes).

These data demonstrate that DSB-(NmG)₂ is a potent in vivo inhibitor of HIV-1 replication. DSB-(NmG)₂ demonstrated potency similar to 3TC in this model.

### SUMMARY

All three salts of DSB exhibited significantly higher water solubility than the free acid (˜100 μg/mL). The NmG salt of DSB was demonstrably more soluble in a solution of 10% hydroxypropyl-β-cyclodextrin in water than both the potassium and sodium salts. The rat bioavailability study evaluated the three salt forms of DSB in five different formulations (three with NmG, one with sodium, and one with potassium) and five different formulations containing the free acid. The NmG salt formulations had the highest oral bioavailability, ranging from 49%-71%. In addition, the SCID-Hu mouse study demonstrated that DSG-(NmG)₂ is a potent in vivo inhibitor of HIV-1 replication.

The foregoing description of the specific embodiments will so fully reveal the general nature of the invention that others can, by applying current knowledge, readily modify and/or adapt for various application such specific embodiments without departing from the generic concept, and therefore such adaptations and modifications are intended to be comprehended within the meaning and range of equivalents of the disclosed embodiments. It is to be understood that the phraseology or terminology herein is for the purpose of description and not of limitation.

All references cited in this specification are hereby incorporated by reference.

What is claimed is:

1. An orally-bioavailable salt of 3-O-(3′,3′-dimethylsuccinyl)betulinic acid comprising two equivalents of N-methyl-D-glucamine per equivalent of 3-O-(3′,3′-dimethylsuccinyl) betulinic acid, wherein said salt has in-vivo anti-HIV activity when administered orally to an HIV-infected mammal.

2. A pharmaceutical composition comprising the salt of claim 1, and a pharmaceutical acceptable carrier or diluent.

3. The pharmaceutical composition of claim 2, wherein said carrier or diluent is an aqueous solvent.

4. The pharmaceutical composition of claim 3, further comprising a cyclodextrin.

5. The pharmaceutical composition of claim 4, wherein the cyclodextrin is hydroxypropyl-β-cyclodextrin.

6. A method of treating HIV infection comprising orally administering to a human patient in need of such treatment, an effective amount of the salt of claim 1.

7. The method of claim 6, wherein said salt is administered in combination with at least one additional active agent selected from an antiviral agent, an immunomodulating agent, an anticancer agent, an antibacterial agent, and an antifungal agent.

8. A method of treating HIV infection comprising orally administering to a human patient in need of such treatment, an effective amount of the pharmaceutical composition of claim 2.

9. The method of claim 8, wherein said pharmaceutical composition is administered in combination with at least one additional active agent selected from an antiviral agent, an immunomodulating agent, an anticancer agent, an antibacterial agent, and an antifungal agent.

10. A method of treating HIV infection comprising orally administering to a human patient in need of such treatment, an effective amount of the pharmaceutical composition of claim 2 in a solid dosage form.

11. The method of claim 10, wherein said pharmaceutical composition is administered in combination with at least one additional active agent.

12. A method of treating HIV infection comprising orally administering to a human patient in need of such treatment, an effective amount of the pharmaceutical composition of claim 2 in a liquid dosage form.

13. The method of claim 16, wherein said pharmaceutical composition is administered in combination with at least one additional active agent.

14. A method of treating HIV infection comprising orally administering to a human patient in need of such treatment, an effective amount of the pharmaceutical composition of claim 2.

15. The method of claim 14, wherein said pharmaceutical composition is administered in combination with at least one additional active agent.

16. A method of preparing the salt of claim 1, said method comprising
mixing N-methyl-D-glucamine and 3-O-(3′,3′-dimethylsuccinyl) betulinic-acid in an aqueous solvent to provide the di-N-methyl-D-glucamine salt of 3-O-(3′,3′-dimethylsuccinyl) betulinic acid.

17. The method of claim 16, wherein said mixing occurs in the presence of hydroxypropyl-β-cyclodextrin.

18. A dosage form comprising the pharmaceutical composition of claim 2.

19. The dosage form of claim 18, wherein said dosage form is a solid oral dosage form, a parenteral dosage form, a liquid dosage form, a suspension dosage form, or a rectal suppository.

20. The dosage form of claim 19, wherein said solid oral dosage form is a tablet.
